(19) United States
(12) Patent Application Publication
(10) Pub. No.: US 2022/0044788 A1
(43) Pub. Date: Feb. 10, 2022

(54) System and Method for Determining Caloric Requirements of an Animal

(71) Applicant: Hill's Pet Nutrition, Inc., Topeka, KS (US)

(72) Inventors: Susan Wernimont, Lawrence, KS (US); Kristen Carnagey, Lawrence, KS (US); Cheryl Stiers, Holton, KS (US); Robin Thompson, Newcastle upon Tyne (GB); Visalakshi Lakshmanan Narayanan, Lawrence, KS (US)

(73) Assignee: Hill's Pet Nutrition, Inc., Topeka, KS (US)

(21) Appl. No.: 17/388,393

(22) Filed: Jul. 29, 2021

Related U.S. Application Data
(60) Provisional application No. 63/060,775, filed on Aug. 4, 2020.

(51) Int. Cl. G16H 20/60; A01K 29/00; A01K 11/00

(52) U.S. Cl. CPC G16H 20/60; A01K 11/008; A01K 29/005

(57) ABSTRACT

A system, apparatus, and/or method of may determine a caloric requirement of an animal. Animal characteristic data including a weight, body type, or breed of the animal may be received. Movement data of the animal may be received (e.g., received via a sensor) for a first predetermined time period. The movement data may include a speed of the animal, a distance traveled by the animal, a location of the animal, and/or steps taken by the animal. An activity level of the animal during the first predefined time period may be determined based on the movement data of the animal. A caloric requirement of the animal during a second predetermined time period may be determined. The caloric requirement of the animal may be based on the activity level of the animal and/or the animal characteristic data of the animal. The caloric requirement of the animal may be displayed.

### CROSS-REFERENCE TO RELATED APPLICATIONS

The present application claims priority to U.S. Provisional Patent Application Ser. No. 63/060,775, filed Aug. 4, 2020, the entirety of which is incorporated herein by reference.

### BACKGROUND

Veterinarians and animal caregivers need to know the appropriate amount of food to feed a cat or dog. Overfeeding (which can result in obesity) and/or underfeeding (which can result in malnourishment) dogs and cats are known, likely due to pet owners providing an amount of food inappropriate to their pet's size, life stage, and activity level. Currently, caloric requirements for cats and dogs are calculated based on a daily caloric requirement that is typically based on published (i.e., static) equations.

Of the characteristics that contribute to the determination of the daily caloric requirement, one the most difficult for animal caregivers and veterinarians to determine is pet activity level. Determining the correct daily caloric requirement typically requires guesses to be made about the animal's level of activity, which is often not observed by pet owners. Ultimately, because of the inability to accurately determine the amount of food needed by a pet, most veterinarians and animal caregivers ultimately feed an amount of food based on a “best guess” and monitor the animal's weight to determine if the guess was correct. While practical, this solution allows for weeks or months of excessive or inadequate feeding before a problem is detected. Further, such solution relies on frequently, and manually, weighing of the pet to measure success.

What is desired is a tool that provides (1) an objective and accurate assessment of pet activity, and (2) provides a near-real time output of that activity would greatly enhance efforts to appropriately manage pet weight. Such a tool may be a system, apparatus, and/or method that uses measurements of the animal's activity to provide energy (and thus food) requirements that are based on an objective assessment of activity.

### BRIEF SUMMARY

A system, apparatus, and/or method of determining a health condition of an animal is provided. In an aspect animal characteristic data comprising at least one of a weight of an animal, a body type of the animal, and/or a breed of the animal may be received. Movement data of the animal may be received (e.g., received via a sensor) for a first predetermined time period, wherein the movement data comprises at least one of the speed of the animal during the first predetermined time period, the distance traveled by the animal during the first predetermined time period, the location of the animal during the first predetermined time period, or the steps taken by the animal during the first predetermined time period. An activity level of the animal during the first predefined time period may be determined based on the movement data of the animal during the first predetermined time period. A caloric requirement of the animal during a second predetermined time period may be determined. The caloric requirement of the animal may be based on the activity level of the animal during the first predetermined time period and the animal characteristic data of the animal. The caloric requirement of the animal during the second predetermined time period may be displayed via a display device.

### BRIEF DESCRIPTION OF THE DRAWINGS

The present invention will become more fully understood from the detailed description and the accompanying drawings, wherein:

FIG. 1 is a block diagram of a system having a plurality of modules configured to collect and analyze the behavior of an animal;

FIG. 2 is a perspective view of an example activity collar;

FIGS. 3A and 3B are depictions of animals wearing an example activity collar;

FIGS. 4A-4D are depictions of an animal wearing an example mechanism on a head of the animal;

FIGS. 5A-5E are depictions of an animal wearing an example mechanism on a tail of the animal;

FIGS. 6A, 6B are depictions of an animal wearing an example mechanism on a limb of the animal;

FIGS. 7A-7D are example screenshots of a use of the system of FIG. 1; and

FIG. 8 is an example process of determining a caloric requirement of an animal, as described herein.

### DETAILED DESCRIPTION

The following description of the preferred embodiment(s) is merely exemplary in nature and is in no way intended to limit the invention, its application, or uses.

The description of illustrative embodiments according to principles of the present invention is intended to be read in connection with the accompanying drawings, which are to be considered part of the entire written description. In the description of embodiments of the invention disclosed herein, any reference to direction or orientation is merely intended for convenience of description and is not intended in any way to limit the scope of the present invention. Relative terms such as “lower,” “upper,” “horizontal,” “vertical,” “above,” “below,” “up,” “down,” “top,” and “bottom” as well as derivatives thereof (e.g., “horizontally,” “downwardly,” “upwardly,” etc.) should be construed to refer to the orientation as then described or as shown in the drawing under discussion. These relative terms are for convenience of description only and do not require that the apparatus be constructed or operated in a particular orientation unless explicitly indicated as such.

Terms such as “attached,” “affixed,” “connected,” “coupled,” “interconnected,” and similar refer to a relationship wherein structures are secured or attached to one another either directly or indirectly through intervening structures, as well as both movable or rigid attachments or relationships, unless expressly described otherwise. Moreover, the features and benefits of the invention are illustrated by reference to the exemplified embodiments. Accordingly, the invention expressly should not be limited to such exemplary embodiments illustrating some possible non-limiting combination of features that may exist alone or in other combinations of features; the scope of the invention being defined by the claims appended hereto.

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

The system, method, and apparatus provide an automatic and accurate amount of calories (e.g., via food sources) required and/or recommended of an animal, such as a pet (e.g., dog, cat, etc.), farm animal (e.g., horse, cow), bird, fish, and the like. The amount of calories required may be based on many factors relating to the animal, such as the activity level of the animal, the movements of the animal, the breed of the animal, the size and/or body type (e.g., long, tall, thin, stocky) of the animal, etc. Veterinarians and animal caregivers (i.e., pet owners or pet parents) may need to know the appropriate amount of calories to provide an animal, in which the calories may be provided via food consumed by the animal. For example, by providing the appropriate amount of food to an animal, the health of the animal may be positively impacted, such as by preventing obesity in the animal or providing nutrition therapy for obesity in the animal. The prevalence of obesity in pets (e.g., dogs and cats) has been identified as a health concern. Such prevalence of obesity in pets suggests that many pet owners are providing the pet an amount of food inappropriate for the pet's size, life stage, activity level, and the like. By providing the appropriate amount of food (e.g., calories) to an animal, the prevalence of health disorders (e.g., disorders caused by obesity) may be decreased or prevented.

Caloric requirements for pets may be determined in one or more ways and/or using one or more parameters. For example, caloric requirements for pets may be determined based on a Daily Energy Requirement (DER) value. The DER value may be calculated as a Resting Energy Requirement (RER) value multiplied by one or more other parameter values. For example, the RER value may be multiplied by a factor F, as shown in Equation 1 below.

DER=RER×F. Equation 1:

The value of RER may be based on the metabolic body weight of a pet and may be based on kcals/day. In examples, metabolic body weight may be weight (e.g., in kilograms), and expressed as KG^{0.75}. An average RER for dogs and cats may be 70 kcals/day (e.g., per KG^{0.75}).

Factor F, which may be used to obtain DER, may incorporate one or more factors related to the animal, such as the animal's activity level, neuter status, life stage, health status, and the like. Factor F may be obtained from one or more lists (e.g., lists containing predetermined values). For example, for neutered adult dogs the factor may be 1.6, for intact adult dogs the factor may be 1.8, for inactive/obese prone dogs the factor may be 1.2-1.4, for dogs in critical care the factor may be 1.0, and for extremely active dogs the factor may be 5.0-11.0. In cats, the factor for neutered adult cats may be 1.2-1.4, for intact adult cats the factor may be 1.4-1.6, for inactive/obese prone cats the factor may be 1.0, and for cats in critical care the factor may be 1.0. The value of the factor may also, or alternatively, vary according to other conditions of the animal, such as a condition (e.g., pregnancy condition) of the animal, whether the animal is lactating, and the like.

Pet activity level may contribute to the choice of a factor for the calculation of DER. Pet activity may be difficult for animal caregivers and veterinarians to determine (e.g., estimate). For example, estimating the correct factor to use may require guesses to be made about the animal's level of activity, which may not be observed by pet owners. Further, activity factors may not be published in ways that reflect several different conditions co-occurring. Thus, a veterinarian or animal caregiver may have to combine factors to represent (e.g., accurately represent) the condition of a pet. For example, a veterinarian or animal caregiver may have to combine factors such as a cat being an obese prone lactating female cat or a dog being a neutered young extremely active male dog. Such combining of factors may require guesswork in conventional systems. Ultimately, because of the inability to accurately estimate the amount of food needed by a pet, veterinarians and animal caregivers may feed an amount of food based on a best guess and monitor the animal's weight to determine if the guess was correct. While guessing strategies may be practical, such solutions allow for weeks or months of excessive or inadequate feeding before a problem is detected and relies on frequently manual weighing of the pet to measure success, which may not be easily performed in many home environments (e.g., may require a visit to a veterinarian). To compound the problem, pet owners and vets may fail to recognize obesity in animals when present. As a result, most pets may not be fed an appropriate amount of food, as evidenced by current rates of pet obesity and being overweight.

Examples herein describe a system, method, and/or apparatus that overcome the limitations in which calorie requirements are assessed for pets. For example, examples herein describe a tool that provides (1) an objective, automatic, and accurate assessment of pet activity and (2) a rapid (e.g., near-real time, such as same day or next day) output of that activity that enhances efforts to appropriately manage pet weight. The tool may use measurements of the animal's activity to provide energy (and thus food) requirements that may be based on an objective assessment of activity. The automatic and accurate determination of caloric requirements of an animal may result in many benefits to the animal, especially if the veterinarian and/or animal's caretaker take corrective action as result of the determined caloric requirements. For example, the weight of the animal may be properly managed, leading to fewer and/or less severe health issues as a result of obesity or the animal being overweight.

The system may include one or more devices and/or mechanisms worn by an animal for receiving, determining, storing, and/or transmitting information of the animal. The mechanisms may be worn on one or more of the head of the animal, the ears of the animal, the neck of the animal, the torso of the animal, limbs (e.g., arms, legs) of the animal, the tail of the animal, the mouth (e.g., tooth, cap over the tooth, replacement tooth), the eye (e.g., contact lenses), and the like. The mechanisms may be placed in one or implants within the animal, such as implants within the belly and/or base of the tail of the animal, a neuticle of the animal, etc. The system may include one more devices coupled to a collar, harness, bracelet, anklet, belt, earring, headband, and the like. In other examples the system may include one or more devices attached to one or more attachment mechanisms, such as a coat, boot, decorative clothing (e.g., ribbon), sweater, hat, etc. In other examples one or more of the devices and/or mechanisms may be implanted within the animal. For example, one or more of the devices and/or mechanisms may be a subdermal implant that may be placed underneath the skin of the animal.

A device (e.g., a recognition device) may identify the animal within the system. The animal may be linked to an animal profile. Movements associated with the animal's profile may be monitored, tracked, and/or electronically recorded on a predefined frequency (e.g., on a daily, weekly, monthly, yearly basis). The animal's movement may be used to determine the animal's activity level, movements, etc., as described herein. The animal's movement may be monitored, tracked, and/or recorded without disturbing the animal or disrupting its natural behavior.

The monitoring of the animal's activity level and/or movement may be performed via collection of one or more types of data. The data may include motion data of the animal (e.g., detected via an accelerometer), location data of the animal (e.g., detected via a global positioning system (GPS) device, proximity beacons, cellular tower triangulations), and the like. The data may be collected and/or monitored during one or more pet activities, such as walking, running, jogging, crawling, trotting, cantering, galloping, ambling, and the like. The data may relate to the animal moving in one or more directions, such as in a forward direction, reverse direction, sideward direction, up/down direction, etc. The data may relate to a gait of the animal, the speed at which the animal is moving, the duration in which the animal is moving, etc. Collected data may be stored in a repository that may be accessible to animal caregivers, veterinarians, and the like. The data may be accessible via a portable electronic device (e.g., an application of a portable electronic device) and/or a server.

A portable electronic device may be one or more of a number of devices, including without limitation, a smart phone, a cell phone, a tablet computer, a personal digital assistant (“PDA”), a laptop computer, etc. The data (e.g., activity level, movement data, location data, etc.) of the animal may be analyzed to determine caloric (e.g., calorie) requirements of the animal. The determined caloric requirements may be provided to veterinarians and/or pet owners based on the data (e.g., activity level, movement data, location data, etc.). The activity level and/or movement data may be collected and/or generated over time, for example, for statistical processing of the animal's caloric requirements. The data may be compared with previously collected and/or stored data for purposes of correlating the animal's activity levels and/or movements with the caloric requirements of the animal. The previously collected and/or stored data may relate to the animal that is being monitored and/or the previously collected and/or stored data may relate to another animal (e.g., an animal with similar characteristics, such as an animal of the same breed, size, body type (e.g., long, tall, thin, stocky, etc.), weight, condition, etc.) for comparison purposes.

An animal's caloric requirements, such as whether the animal is consuming too little, just enough, or too many calories, may be determined based on the activity level and/or movement of the animal. The animal's caloric requirements may be recorded. To determine the animal's calorie requirements, parameters indicative of the animal's movements and/or activity levels may be monitored and/or recorded. Such parameters may include the speed, acceleration, duration, distance, direction, etc. that an animal walks, runs, jogs, gallops, rests, etc. As an example, an animal running multiple hours per day may have a higher caloric requirement than an animal (e.g., an animal with similar characteristics) that runs less than one hour per day. As described herein, the caloric requirement may be based on attributes other than the activity level of the animal, such as the breed of animal, sex of animal, size and/or body type of animal, pregnancy status of the animal, etc. For example, a pregnant animal and/or a large animal that runs less than an hour a day may have a higher caloric requirement than a non-pregnant animal and/or a small animal (e.g., an animal with different characteristics) that runs more than an hour a day.

The application of statistical methods may be used to derive information about the animal's caloric requirements based on the activity level and/or movement of the animal. For example, an animal running an amount of time (e.g., a minimum and/or maximum amount of time) during a time period (e.g., per day, per week, per month, etc.) may be expected to have a predetermined caloric requirement during a future time period that may be the same or different than the time period in which the animal is monitored and/or for which the caloric requirement is determined. If parameters related to the animal deviate from a defined parameter (e.g., the animal runs for less than, or more than, an amount defined for the animal), it may be determined that the animal may require less calories or more calories than the caloric requirement previously predetermined for the animal. In other examples, the body fat index (BFI), body condition score (BCS), muscle condition score (MCS), and/or weight of the animal may be determined and/or identified to derive the caloric requirement of the animal. The BFI, BCS, MCS and/or weight of the animal may be determined as larger animals moving the same distance at the same gait as smaller animals may require more calories to move a larger body mass than a smaller body mass.

As described herein, subsets of characteristics of the animal may be used to determine caloric requirements of the animal. Such characteristics may include the specie, breed, size, body type, age, sex, geographic location, and/or size/weight of the animal. For example, a dog may be expected to move faster and/or farther than a cat. As a result, a running distance per day that substantially increases the caloric requirements for a cat may not similarly substantially increase the caloric requirements for a dog running a similar distance per day.

Parameters determined, identified, received, and/or transmitted may be recorded. The parameters may be recorded continuously, for example, from the moment of system activation throughout animal's life. In other examples, the parameters may be recorded for a predefined time period (e.g., for a day, a week, a month, etc.), on a predefined frequency (e.g., every weekday), etc.

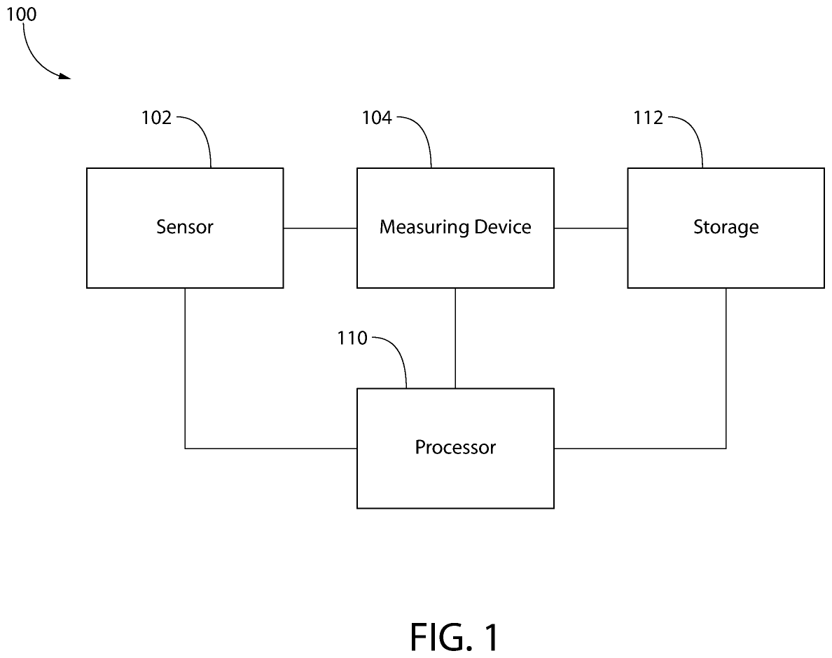

FIG. 1 shows an example system for determining caloric requirements for an animal based on the activity level and/or movements of the animal. System 100 may include a sensor 102, a measuring device 104, and/or a storage device 112.

Sensor 102 may be configured to detect the motion (or stillness) of an animal, to detect an activity level of the animal, to detect an orientation of the animal, to detect a location of the animal, etc. Sensor 102 may be one or more of a variety of form factors, including, but not limited to, an accelerometer, a gyroscope, a magnetometer, force transducers, displacement transducers, pressure transducers, force sensors, displacement sensors, pressure sensors, load cells, photographic cameras, video cameras, camcorders, and a combination thereof. In examples, sensor 102 may include one or more of thermometers, electrocardiography (ECG), photo plethysmography (PPG) devices, microphones, respiratory inductive plethysmography (RIP) devices, optoelectronic plethysmography (OEP) devices, or transthoracic impedance devices. For example, caloric expenditure may be assessed by heat produced, by cardiac/respiratory output, distance traveled, and/or step metrics. ECG and PPG may provide pulse/heart rate detection. Microphones, RIP, OEP and impedance may provide a breathing rate.

In addition, or alternatively, sensor 102 may be one or more of optical sensors, optical reflecting sensors, LED/photodiode pair optical sensors, LED/phototransistor pair optical sensors, laser diode/photodiode pair optical sensors, laser diode/phototransistor pair optical sensors, optocouplers, optical fiber coupled optical sensors, magnetic sensors, weight sensors, force sensors, displacement sensors, pressure sensors, various proximity sensors, such as inductive proximity sensors, magnetic proximity sensors, capacitive proximity sensors, and/or a combination thereof. Sensor 102 may include communication circuitry, such as Bluetooth (e.g., classic Bluetooth and/or Low Energy Bluetooth), RFID, Wi-Fi, and other wireless technologies. Sensor 102 may communicate with one or more devices, for example, sensor 102 may communicate with a server.

Measuring device 104 may be configured to measure a characteristic related to the animal. Measurement device 104 may be a device that is separate from sensor 102 or a device that is the same as sensor 102. Example measuring devices 104 may be implemented in one or more of a variety of form factors, including, but not limited to, weighing scales, weight transducers, force transducers, displacement transducers, pressure transducers, weight sensors, force sensors, displacement sensors, pressure sensors, real time clocks, timers, counters, and/or a combination thereof. Measuring device 104 may include communication circuitry, such as blue tooth, RFID, Wi-Fi, and other wireless technologies. Measuring device 104 may communicate with one or more devices, for example, measuring device 104 may communicate with a server.

Storage device 112 may be configured to store data provided to and/or from system 100. The data may include motion data, activity level data, location data, temperature data, heart rate data, respiratory (breathing) rate data, weight data, mass data, as described herein. The data may be provided by the sensor 102. Example storage devices 112 may be memory devices, data storage devices, and a combination thereof, such as memory chips, semiconductor memories, Integrated Circuits (IC's), non-volatile memories or storage device such as flash memories, Read Only Memories (ROM's), Erasable Read Only Memories (EROM's), Electrically Erasable Read Only Memories (EEROM's), Erasable Programmable Read Only Memories (EPROM's), Electrically Erasable Programmable Read Only Memories (EEPROM's), an Electrically Erasable Programmable Read Only Memory (EEPRO), volatile memories such as Random Access Memories (RAM's), Static Random Access Memories (SRAM's), Dynamic Random Access Memories (DRAM's), Single Data Rate memories (SDR's), Dual Data Rata memories (DDR's), Quad Data Rate memories (QDR's), microprocessor registers, microcontroller registers, CPU registers, controller registers, magnetic storage devices such as magnetic disks, magnetic hard disks, magnetic tapes, optical memory devices such as optical disks, compact disks (CD's), Digital Versatile Disks (DVD's), Blu-ray Disks, Magneto Optical Disks (MO Disks) and/or a combination thereof. In one embodiment, the storage device comprises a semiconductor RAM IC for an intermediate recording of the behavior, health, and/or characteristics of the animal, and then transfer of the data to a flash memory IC for non-volatile recording. Storage 112 may be an external memory device, such as a USB flash memory, an external hard drive, etc.

System 100 may include a processor 110 configured to calculate and/or process data provided to system 100. For example, processor 110 may be configured to determine the calories required of an animal based on an activity level, as described herein. Example processors may be electronic circuits, systems, modules, subsystems, sub modules, devices, and combinations thereof, such as Central Processing Units (CPU's), microprocessors, microcontrollers, processing units, control units, tangible media for recording and/or a combination thereof. Storage device 112 may be configured to store derived data from the processor 110. Processor 110 may include communication circuitry, such as blue tooth, RFID, Wi-Fi, Medical Implant Communication System (MICS) (e.g., a hybrid of the technologies, such as MICS/Bluetooth), and other wireless technologies. Processor 110 may communicate with one or more devices, for example, processor 110 may communicate with a server.

In an example, sensor 102, and/or storage 112 may be assembled in a number of configurations, including in a stand-alone apparatus. In another example, one or more of sensor 102, storage 112, and processor 110 may each be assembled in a stand-alone apparatus. In other examples, one or more of the processor 110 and/or storage 112 may be configured as remote devices, such as remote servers (e.g., cloud storage devices). Although FIG. 1 shows a connection between processor 110 and each of sensor 102, measuring device 104, and storage 112, examples should not be so limited. In examples one or more of the devices may communicate with one or more (including any, or none) of the other devices. For example, sensor 102 may communicate with processor 110 and storage 112, sensor 102 may not communicate with storage 112, etc. One or more devices may be added and/or removed from system 100. For example, additional sensors 102 may be added to system 100 and/or storage 112 may be removed from system 100.

Data relating to the animal may be processed and/or recorded for a determination of the animal's caloric requirements. For example, the amount of times, durations, speed, directions, etc., in which an animal walks, jogs, runs, and/or rests may be used to determine an activity level of the animal and/or the caloric requirements of the animal, etc. A weight of an animal, a body temperature of an animal, a size and/or body type of an animal, a breed of an animal, the date and/or time of an event (e.g., a walking, running of the animal) relating to an animal, etc. may be used to determine caloric requirements of an animal. One or more activities of the animal may be recorded via a video recording, picture, audio recording, thermal imaging, etc. The recording of the animal activities may be processed.

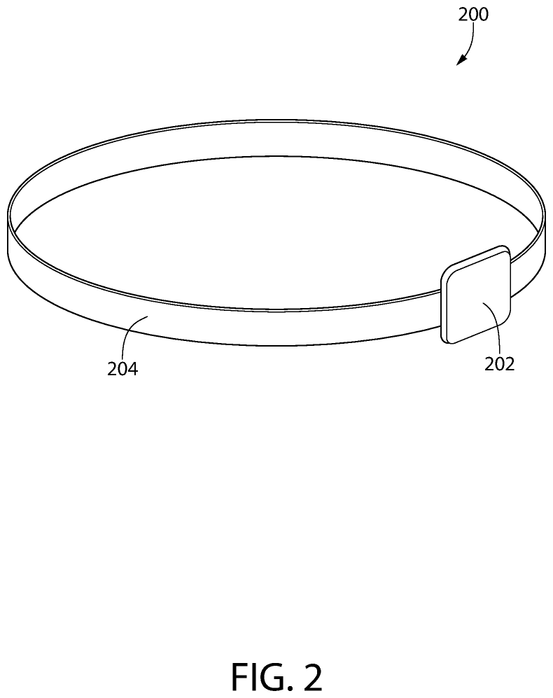

FIG. 2 is a perspective view of an example mechanism 200 worn by an animal. Although FIG. 2 shows mechanism 200 as a collar, it should be understood that mechanism 200 may be one or more mechanisms worn by an animal and/or constraining the animal. Mechanism 200 may be worn on one or more of the head of the animal, the ears of the animal, the neck of the animal, the torso of the animal, limbs (e.g., arms, legs) of the animal, the tail of the animal, and the like. For example, mechanism 200 may include a collar, harness, bracelet, anklet, belt, earring, headband, and the like. In other examples devices that may house or couple to an electronic device may include one or more attachment mechanisms, such as a coat, boot, decorative clothing (e.g., ribbon), sweater, hat, etc. Mechanism 200 may be used to constrain the animal, store information about the animal, process information about the animal, and/or transmit information relating to the animal.

Mechanism 200 may be linked to a particular animal (e.g., may be linked to a profile of a particular animal). Mechanism 200 may include circuitry 202 that may include a processor, storage, wireless communication hardware, one or more sensors (e.g., accelerometers, gyroscopes, magnetometers, etc.), GPS, temperature sensors, moisture detectors, biometric sensors, etc. As described herein, mechanism and/or sensor may include one or more of a thermometer, microphone, speaker, electrocardiography (ECG) device, photo plethysmography (PPG) device, microphones, respiratory inductive plethysmography (RIP) device, optoelectronic plethysmography (OEP) device, or transthoracic impedance device. The wireless communication hardware may include a transmitter and a receiver. For example, the wireless communication hardware of the mechanism 200 may include a low energy communication device, such as Bluetooth Low Energy or RFID. The wireless communication hardware may include Bluetooth, Medical Implant Communication System (MICS), or a hybrid of the technologies, such as MICS/Bluetooth. The mechanism 200 may include a memory for storing data.

An accelerometer located on the mechanism 200 may be configured to measure one or more motions of the animal. For example, the accelerometer may measure accelerations of the animal, changes in velocity of the animal, and/or changes in position of the animal. A gyroscope may be configured to measure changes in orientation of the animal and/or changes in rotational velocity of the animal. A magnetometer may be configured to measure orientation (e.g., absolute orientation) of the animal, for example, in the NESW plane.

As described above, the mechanism 200 may include a GPS. The GPS may track a position of the animal. For example, the GPS may indicate that the animal is inside a home, outside a home, etc. For example, the GPS may indicate that the animal is near a food or drinking area (e.g., food bowl, drinking bowl, etc.), within a park (e.g., a dog park), within an exercise area (such as an exercise area of a dog boarding kennel), within a crated area, etc. The movement of the animal may be associated with the location of the animal. For example, the caloric requirements of an animal may be increased if the animal is running outside of the home.

Mechanism 200 may send data relating to an animal to a server, electronic device (e.g., mobile phone of pet parent or caregiver), and the like. For example, mechanism 200 may send motion data, orientation data, location data, etc., to a server, electronic device, etc. The server may perform computations of the data, for example, to determine the present and/or future caloric requirements of the animal based on factors described herein, such as the present and/or past activity levels of the animal or movements of the animal. The server may store previous caloric requirements determined for the animal, predict present and/or future caloric requirements for the animal (assuming the animal will have the same or similar activity level and/or movements as the animal had in the past).

The determined caloric requirements for the animal may be compared with other animals that may be similarly situated (e.g., similar breed, similar size, similar body type, similar activity level, etc.). The server may determine how accurate the determined caloric requirements were for the other animals, for example, based on how effective the determined caloric requirements were for correcting health disorders (such as obesity). Based on how accurate the determined caloric requirements were for the other animals, the server may perform adjustments for the determination of the animal. The server may be configured to communicate the data to the user and/or to one or more other parties (e.g., veterinarians, pet parents, care givers, etc.). In examples, an electronic device (e.g., the care giver's mobile phone) may perform computations of the data to determine caloric requirements of the animal. The electronic device may be configured to communicate the data to the user and/or one or more other parties (e.g., a veterinarian, spouse, etc.).

The mechanism 200 may have a biometric monitoring sensor. The biometric monitoring sensor may be configured to determine body measurements and/or calculations of the animal. For example, temperature sensor and/or heart rate sensor may be used to determine the body temperature of the animal and/or the heart rate of the animal. As an example, caloric expenditure may be assessed by heat produced, by cardiac/respiratory output, distance traveled, and/or step metrics. An ECG and/or PPG may provide pulse/heart rate detection. In other examples the respiration rate of the animal and/or the glucose level (e.g., via a glucose sensor) may be determined. Microphones, RIP, OEP and/or an impedance may provide a breathing rate.

The biometric monitoring sensor may be located on the activity collar or on another device position on or about the animal. The biometric data may be used to determine caloric requirements of the animal.

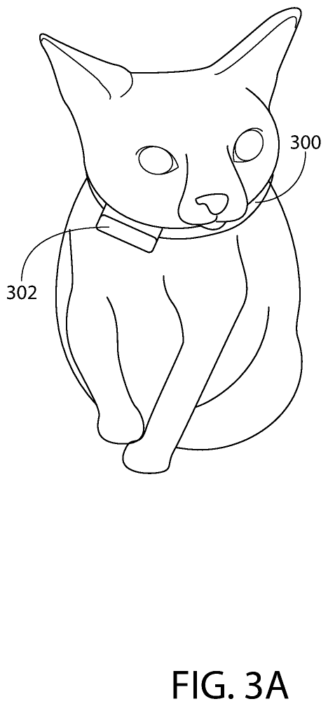

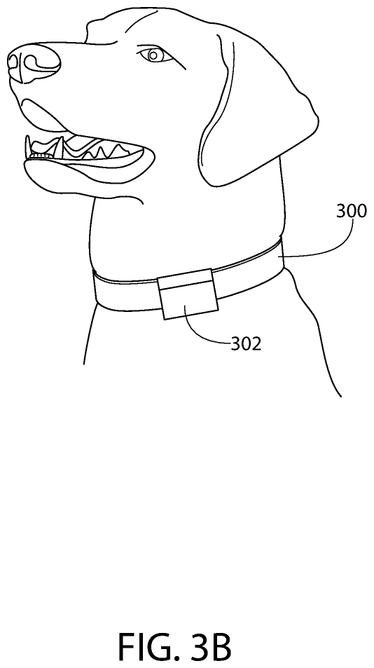

FIGS. 3A, 3B show examples of mechanism 300. As shown on FIG. 3A, a cat may wear the mechanism 300 in the form factor of an activity collar, in other examples other animals can wear mechanism 300, such as the dog shown wearing the activity collar in FIG. 3B. Although the example mechanism 300 as shown on FIGS. 3A/3B is a collar, it should be understood that the collar is for illustration purposes only and mechanism 300 may be any device (e.g., wearable device) that may come in other form factors besides a collar, as described herein. For example, mechanism 300 may be a harness, jacket, vest, hat, gloves, contact lenses, rings (e.g., earrings), chip (e.g., electronic chip), or any other device (or combinations of devices) that can be worn on the outside (or inside) of an animal.

As described herein, the mechanism 300 (e.g., activity collar) may have one or more sensors 302, as described herein. The sensor 302 may be coupled to the mechanism 300, for example, on an outside of the mechanism 300. In other examples, the sensor (e.g., accelerometer) may be integrally formed within the mechanism 300. In yet other examples, sensor 302 may be implanted within the animal, such as a subdermal implant. Sensor 302 may include a location sensor or one or more of the other sensors described herein. The sensor may be located on the animal (e.g., worn by the animal) or positioned upon a surface that is not the animal.

The sensors and other devices may be used to determine the activity level and/or movement of the animal, such as the direction, speed, acceleration, duration, etc. in which the animal is moving. The activity level and/or movement of the animal may be determined based on motion data, orientation data, location data, etc., of the animal. The sensors and other devices may be used to determine the location at which an animal is moving.

Mathematical and/or algorithmic techniques, such as bivariate, multivariate and trend analysis, may be used to formulate a trend of the animal events (e.g., activity level and/or movement of the animal). Data collected over time and processed can represent a typical profile of behavior of an animal. The behavior and habits of the animal may be used to determine the animal's caloric requirements. For example, an animal that frequently exhibits a high activity level may require more calories than an animal that exhibits a burst of high activity after a period of low activity. Also, or alternatively, trend analysis may be used to determine whether the monitored behavior, habits, etc. of the animal are random, or whether a trend may be developing.

Notifications may be delivered to portable electronic device, for example, in the form of an electronic mail message sent to a user-specified electronic mail address, a text message sent via SMS (Short Message Service) to a user-specified mobile phone number, a calendar reminder set up by the system in a user-specified calendar, phone calls to a user-specified mobile or landline phone number, messages via a mobile device (e.g., phone, tablet) application (“app”), etc.

The time and/or duration of an animal's movements may be recorded. For example, a spike (or reduction) in the amount of activity level of an animal may be recorded and sent to the pet owner so that the pet owner may ensure that the pet consumes (e.g., timely consumes) an amount of calories commensurate with the animals activity level. All records may be stored and/or may be presented, for example, via a textual or graphical format.

A profile of the animal may be accessed via a portable electronic device. The portable electronic device may provide a user interface, for example, via an application downloaded on the portable electronic device. A user may create a profile associated with the animal. The application may display the animal's profile and/or may be facilitate the uploading of monitoring information of the animal. Icons or symbols displayed on the application may designate an activity level, movement, or caloric requirement of an animal that is being monitored and/or tracked. Such data may be displayed in graph form for ease of reference.

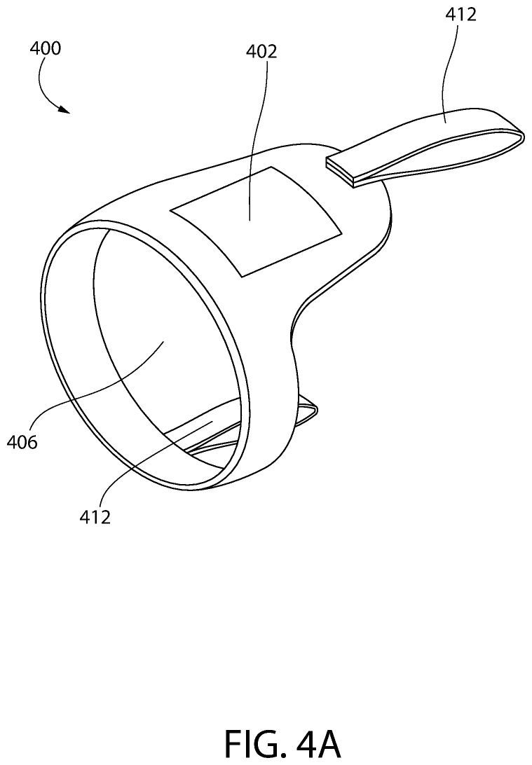

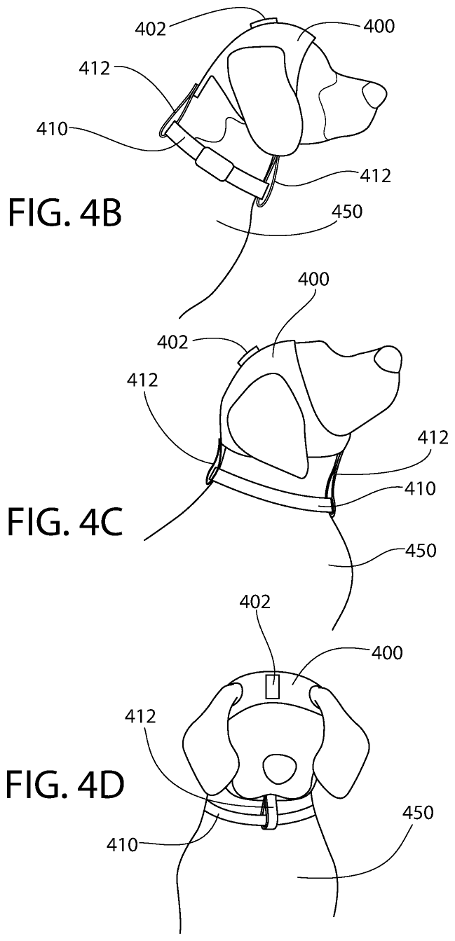

FIGS. 4A-4D show an example mechanism 400. FIG. 4A shows the mechanism 400 alone, and FIGS. 4B-4D show mechanism 400 worn on or about a head of the animal 450. Although FIGS. 4B-4D show mechanism worn on a dog, mechanism 400 may be worn on one or more types of other animals, such as a cat, horse, cow, bird, fish, and the like. Mechanism 400 may include an opening 406 in which a portion, such as the nose, mouth, and/or eyes of the animal may be inserted. Mechanism 400 may be formed around one or more ears of the animal, around the head and/or neck of the animal, and the like. Mechanism 400 may be pulled on an animal, fastened upon an animal, and the like. For example, mechanism 400 may be fastened around the animal using any known fastening device(s), such as a hook, hook and loop, adhesive, string, and the like.

Mechanism 400 may be a standalone device in examples, although in other examples the mechanism 400 may be coupled to one or more devices, such as a collar 410 (FIGS. 4C, 4D). Mechanism 400 may be coupled to a collar 410 via one or more devices, such as strap 412. Mechanism 400 may perform all operations in examples, although in other examples mechanism 400 may share operations with one or more devices, such as a collar 406.

Mechanism 400 may have one or more sensors 402, such as an accelerometer. Sensor 402 may be similar to one or more sensors described herein, such as sensor 300. The sensor 402 may be coupled to the mechanism 400, for example, on an outside of the mechanism 400, under the mechanism 400, and the like. Sensor 402 may be removable and/or chargeable. Although FIGS. 4B-4D show sensor 402 along a center-top portion of mechanism 400, it should be understood that such depiction is for illustration purposes only. In examples sensor 402 may be on top of mechanism 400, on the bottom of mechanism 400, along the sides of mechanism 400, along the borders of mechanism 400, and the like. Sensor 402 (e.g., accelerometer) may be integrally formed within the mechanism 400. Sensor 402 may include a location sensor. The location sensor may be located on the animal (e.g., worn by the animal) or positioned upon a surface that is not the animal. The location sensor may determine the absolute location of the animal or a relative location of the animal.

As described herein, the sensors and other devices may be used to determine the activity level and/or movement of the animal, such as the direction, speed, acceleration, duration, etc. in which the animal is moving. The activity level and/or movement of the animal may be determined based on motion data, orientation data, location data, etc., of the animal. The sensors and other devices may be used to determine the location at which an animal is moving.

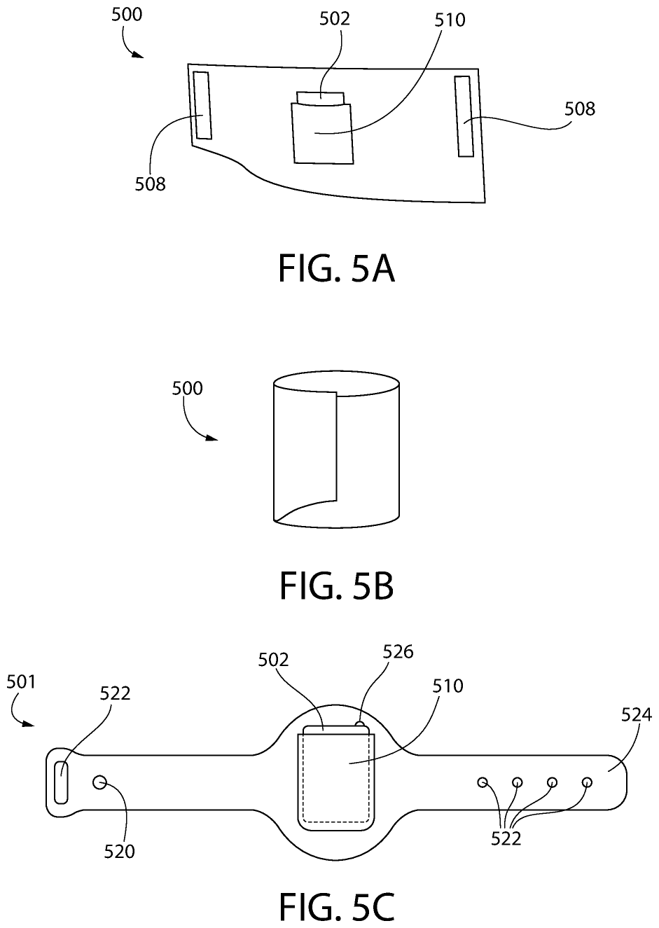

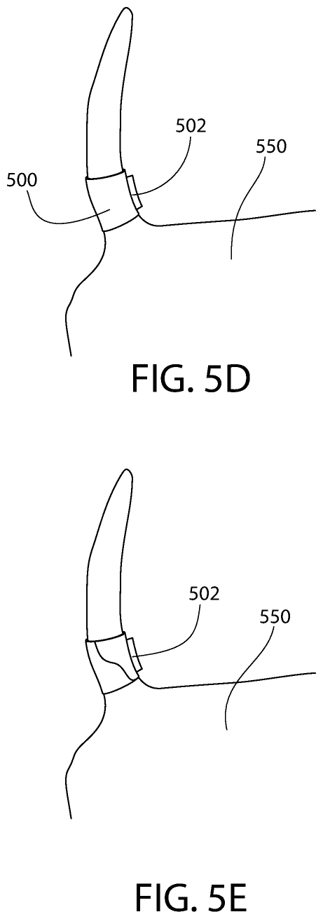

FIGS. 5A-5E show example mechanisms 500, 501. FIGS. 5A-5B show the mechanism 500 alone, and FIGS. 5D, 5E show mechanism 500 worn on a tail of the animal 550. Mechanism 500, 501 may be pulled on an animal (e.g., a tail of an animal), fastened upon an animal, and the like. For example, mechanism 500, 501 may be fastened around the tail of the animal using any known fastening device(s) 508, such as a hook, hook and loop, adhesive, string, and the like. FIGS. 5A, 5B show mechanism 500 with a sleeve 510 for accepting a sensor. Mechanism 500 may include a hook and loop device 512 for fastening the mechanism 500 to the animal.

Mechanism 500 may fold around a portion of the animal, curve around the portion of the animal, etc. As shown in FIG. 5C, mechanism 501 may include sleeve 510 and one or more fastening devices, such as metal pins 520 and one or more openings 522. Openings 522 may be used to receive metal pin 520, although in examples opening 522 may receive one or more connectors (such as strap 524) for fastening mechanism 500 to animal 550. In examples, mechanism 500 may include one or more indicators, such as light 526, battery indicator, and the like. The mechanism 500, 501 may be a standalone device in examples, although in other examples the mechanism 500, 501 may communicate with one or more devices, such as a collar, head attachment, harness, tail attachment, and/or limb attachment. Mechanism 500, 501 may perform all operations in examples, although in other examples mechanism 500, 501 may share operations with one or more devices, such as a collar. FIG. 5C shows mechanism 501 alone. Although FIGS. 5D, 5E show mechanism 500 worn on a tail of a dog, mechanisms 500, 501 may be worn on one or more types of other animals, such as a cat, horse, cow, bird, fish, and the like.

Mechanism 500, 501 may have one or more sensors 502, such as an accelerometer. Sensor 502 may be similar to one or more sensors described herein, such as sensor 300, 400. The sensor 502 may be coupled to the mechanism 500, 501, for example, on an outside of the mechanism 500, 501, under the mechanism 500, 501, and the like. In examples, the sensor 502 (e.g., accelerometer) may be integrally formed within the mechanism 500, 501. Sensor 502 may include a location sensor. The location sensor 502 may be located on the animal (e.g., worn by the animal) or positioned upon a surface that is not the animal. The location sensor may determine the absolute location of the animal or a relative location of the animal.

As described herein, the sensors and other devices may be used to determine the activity level and/or movement of the animal, such as the direction, speed, acceleration, duration, etc. in which the animal is moving. The activity level and/or movement of the animal may be determined based on motion data, orientation data, location data, etc., of the animal. The sensors and other devices may be used to determine the location at which an animal is moving.

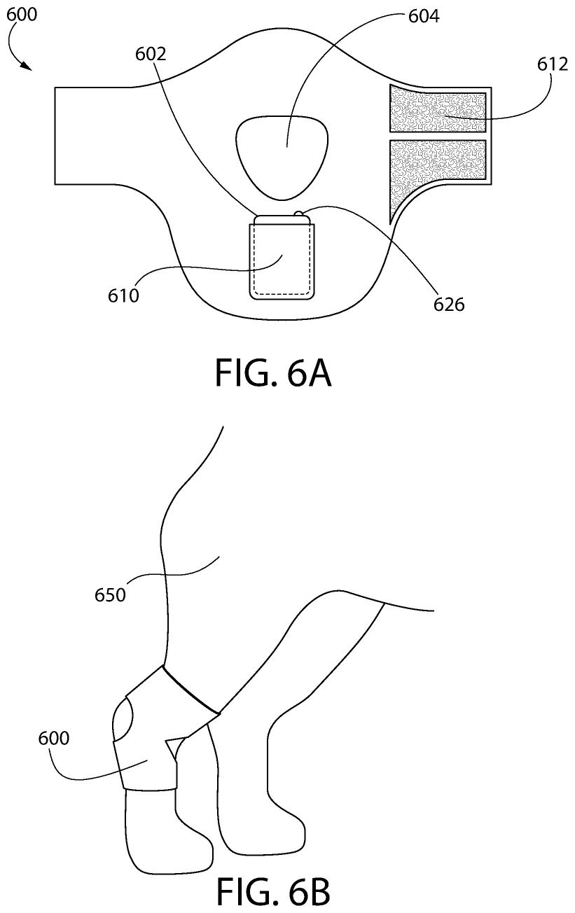

FIGS. 6A, 6B show an example mechanism 600. FIG. 6A shows the mechanism 600 alone, and FIG. 6B show mechanism 600 worn on a limb (e.g., leg) of the animal 650. Mechanism 600 may be pulled on an animal, fastened upon an animal, and the like. For example, mechanism 600 may be fastened around the limb of the animal using any known fastening device(s) 612, such as a hook, hook and loop, adhesive, string, and the like. Mechanism 600 may fold around a portion of the animal, curve around the portion of the animal, etc. Mechanism 600 may include an opening 604 to receiving one or portions of an animal, such as an elbow, wrist, knee, hock, and the like.

FIGS. 6A, 6B show mechanism 600 with a sleeve 610 for accepting a sensor 602. Although FIG. 6A shows sensor 602 on a bottom-center of the mechanism 600, it should be understood that such depiction is for illustration purpose only and the sensor 602 may be positioned on one or more locations of the mechanism 600, such as on the inside, outside, top, bottom, and/or center of mechanism 600. Mechanism 600 may include one or more indicators, such as light 626, battery indicator, and the like. The mechanism 600 may be a standalone device in examples, although in other examples the mechanism 600 may communicate with one or more devices, such as a collar, head attachment, tail attachment, and the like. Mechanism 600 may perform all operations in examples, although in other examples mechanism 600 may share operations with one or more devices, such as a collar. FIG. 6A shows mechanism 600 alone. Although FIG. 6B shows mechanism 600 worn on a limb of a dog, mechanism 600 may be worn on one or more types of other animals, such as a cat, horse, cow, bird, fish, and the like.

As described herein, mechanism 600 may have one or more sensors 602, such as an accelerometer. Sensor 602 may be similar to one or more sensors described herein, such as sensor 300, 400. In examples, the sensor 602 (e.g., accelerometer) may be detachably coupled to the mechanism 600 or integrally formed within the mechanism 600. Sensor 602 may include a location sensor. The location sensor may be located on the animal (e.g., worn by the animal) or positioned upon a surface that is not the animal. The location sensor may determine the absolute location of the animal or a relative location of the animal.

As described herein, the sensors and other devices may be used to determine the activity level and/or movement of the animal, such as the direction, speed, acceleration, duration, etc. in which the animal is moving. The activity level and/or movement of the animal may be determined based on motion data, orientation data, location data, etc., of the animal. The sensors and other devices may be used to determine the location at which an animal is moving.

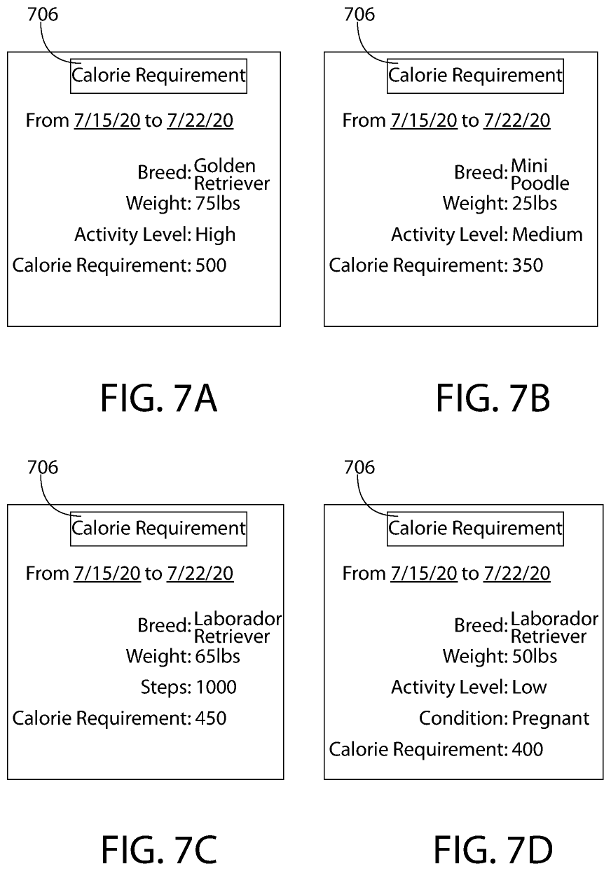

FIGS. 7A-7D show example screenshots of a system providing calorie recommendations of an animal. The screenshots may be provided on a portable electronic device, for example. The screenshots may provide indications of one or more parameters related to the animal, such as the breed, weight, size, body type, activity level, movement (e.g., steps), and conditions (e.g., pregnancy condition) of the animal. The parameters and caloric requirements shown on the screenshots are for illustration purposes only and are not limiting. In examples, other parameters and/or other requirements (e.g., water requirements) may be provided to the user.

FIG. 7A shows an example screen shot of data collected by an activity collar (such as mechanism 200) worn by an animal for determining a calorie requirement. Category 706 shows the Calorie Requirement is being determined for an animal. In other examples, category 706 may show other categories, including the identity (e.g., name) of the animal. The screenshots may be provided on a display, such as on a display of a portable electronic device.

As shown on FIGS. 7A-7D, a time period may be provided. Time period may define the period of the data provided (e.g., the period in which the animal's activity level or movements have been monitored and/or determined). Using the example shown on FIG. 7A, the breed, weight, activity level, and/or caloric requirement is provided for the time period from Jul. 15, 2020 to Jul. 22, 2020. The caloric requirement may be the daily required caloric requirement. For example, as shown on FIG. 7A, the daily required calories required for the animal would be 500 calories. As described herein, time period may be any time period, including a single day (e.g., Jul. 15, 2020). Based on the Jul. 15, 2020 to Jul. 22, 2020 time period provided on FIG. 7A, the breed 720 of the animal is a golden retriever, the weight of the animal is 75 pounds, the activity level of the animal is high, and caloric requirement (e.g., daily caloric requirement) of the animal is 500 calories. Of course, the numbers of these events, and the listing of the events, is for illustration purposes only. For example, the parameter activity level of the animal may be based on a different scale (e.g., may be based on a number scale), may be more or less precise, etc. Different (including more or less) categories of data, time periods, etc., may be displayed.

Using the example shown on FIG. 7B, category 706 may relate to caloric requirements. The caloric requirements may be based on a time period, for example, from Jul. 15, 2020 to Jul. 22, 2020. As described herein, in other examples time period may be any time period, including a single day (e.g., Jul. 15, 2020). Based on the desired time period, the caloric requirements may be provided, including the animal being a miniature poodle, weighing 25 pounds, having an activity level of medium, and a caloric requirement (e.g., daily caloric requirement) of 350.

As shown on FIG. 7C, category 706 may relate to a caloric requirement based on steps taken by the animal (i.e., the number of steps of animal). The steps taken by the animal may be used to determine the distance traveled by the animal, calories burned by the animal, the respiratory rate of the animal, the heart rate of the animal, etc. The steps taken by the animal may influence the calculation of a caloric requirement of the animal. Caloric requirement data may be provided on a time period, as described herein. Based on the desired time period, the caloric requirement may be provided. The caloric requirement may provide animal-specific information, which may include the breed of the animal (e.g., the animal being a Labrador retriever), the weight of the animal (e.g., the animal weighing 65 pounds), the amount of steps taken by the animal (e.g., the animal taking 1,000 steps), and/or the caloric requirement of the animal (e.g., the animal having a caloric requirement (e.g., daily caloric requirement) of 450).

As shown on FIG. 7D, category 706 may relate to a caloric requirement based on an activity level and a pregnancy condition of the animal. Caloric requirements may be provided on a time period, as described herein. Based on the desired time period and one or more other parameters, the caloric requirement of the animal may be provided. For example, the animal being a Labrador retriever, being 50 pounds, having a low activity level, and a pregnancy condition of yes, may result in the animal having a caloric requirement of 400.

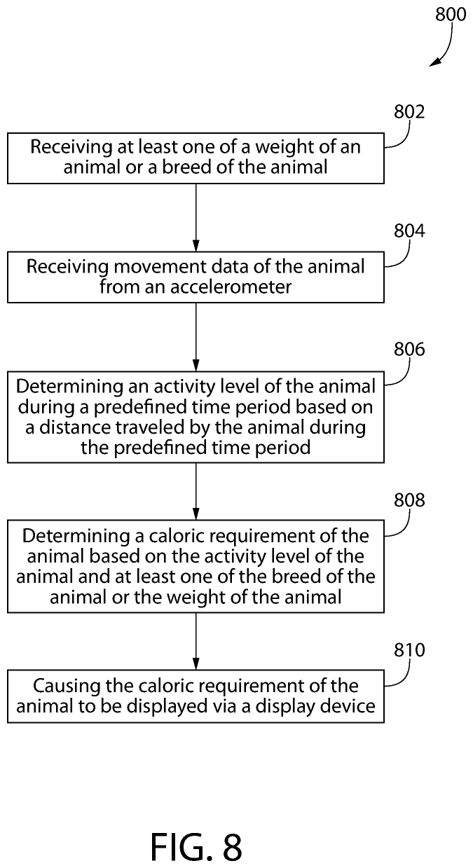

FIG. 8 describes an example method 800 of determining an amount of calories required of an animal, such as a dog or a cat. The amount of calories required by the animal may be based on one or more parameters relating to the animal, such as the activity level of the animal, the movement of the animal, the weight of the animal, the size and/or body type of the animal, the breed of the animal, a condition (e.g., lactation or pregnancy condition) of the animal, etc. At 802, data relating to an animal may be received. For example, data relating to the breed of the animal, the weight of the animal, the size and/or body type of the animal, a condition (e.g., pregnancy or lactating condition) of the animal, may be received. The data may be received by and/or provided to a server and/or a user device (e.g., a user device of a pet owner or a veterinarian).

At 804, movement data of the animal may be received. The movement data of the animal may relate to motion data of the animal, location data of the animal, orientation data of the animal, etc. The movement data of the animal may be the steps taken by animal as the animal walks, runs, gallops, etc. The movement data of the animal may be determined via one or more sensors, such as one or more sensors configured to detect the motion (or stillness) of the animal (e.g., via an accelerometer), to detect a location of the animal (e.g., via a GPS), to detect an orientation of the animal, etc. As described herein, the sensor may be one or more of a variety of form factors, including, but not limited to, an accelerometer, a gyroscope, a magnetometer, photographic cameras, video cameras, camcorders, and a combination thereof.

At 806, an activity level of the animal may be determined. The activity level may be determined for a predetermined time period. The activity level of the animal may be determined based on one or more (including combinations of) movement characteristics of the animal. For example, the activity level of the animal may be determined based on one or more of the speed at which the animal has moved at one or more time periods, the steps that the animal has taken within the predetermined time period, the direction in which the animal has moved, the distance traveled by the animal during the predetermine time period, the duration in which the animal was active, etc. As an example, the activity level of the animal may be based on the distance traveled by the animal at a speed during a predetermined time period, the level of cardiac or respiratory exertion by the animal, the thermal energy generated by the animal, and the like. The distance traveled by the animal may be determined by identifying the number of steps taken by the animal, the speed at which the animal has moved (e.g., the speed at which the animal has moved in concert with the number of steps taken), the direction in which the animal has moved, etc.

At 808, the calories exerted by the animal (e.g., exerted by the animal within a predetermined time period) may be determined. The calories exerted by the animal may be based on the received data (e.g., data relating to the breed of the animal, data relating to the size and/or body type of the animal, etc.), as described herein (e.g., as described in step 802). The calories exerted by the animal may also, or alternatively, be based on the movement data of the animal, such as the activity level of the animal. For example, the distance traveled by the animal may be converted to the calories exerted by the animal via a multiplier, as described herein. The multiplier may be calculated based on the weight of the animal and the activity level of the animal. By determining the calories exerted by the animal, it may be determined the amount of calories that the animal requires (e.g., to replace the calories lost by the animal). For example, the calories required for a defined future time period may be based on the amount of calories exerted by the animal during a previous time period. The previous time period and the future time period may be the same amount of time or a different amount of time.

To determine the calories required of an animal, the resting energy requirement (RER) of the animal may be determined. The RER may be the amount of calories exerted by an animal at rest. The calories (e.g., total calories) exerted by the animal may include the calories exerted while the animal rests and the calories exerted while the animal is active. The RER for the animal may be determined by multiplying the animal's body weight in kilograms (W), raised to the ¾ power, by 70, as provided in Equation 2.

RER=70×W^{0.75} Equation 2:

For animals weighing between 2 kilograms and 45 kilograms, the RER equation may be modified, as provided in Equation 3.

RER=70+(30×W) Equation 3:

The calories exerted (e.g., expended) by the animal may be based on one or more parameters. For example, the calories exerted (e.g., expended) by the animal may be based on the resting energy requirement (RER), as described herein. One or more parameters may be determined based on the RER. For example, the calories exerted (E) may be determined based on the RER, a gait coefficient (G) of the animal, and/or the distance traveled (D) by the animal. Equation 4 shows an example equation for determining the calories exerted (E) from the animal for a time period. Although Equation 4 describes gait coefficient, it should be understood that the use of gait coefficient as a parameter is for illustration purposes only. In examples gait coefficient G may be substituted for one or more other parameters, such as the activity of the animal, the cardiac or respiratory exertion of the animal, the thermal energy generated by the animal, and the like. The parameter (e.g., the gait coefficient) may be multiplied by the distance traveled by the animal to obtain an activity factor. For example, a gait coefficient may correspond to a distance traveled by the animal (e.g., the distance traveled while the animal is moving via the particular gait). An activity level of the animal may be based on the activity factor.

E=RER+(G×D) Equation 4:

The gait coefficient (G) may be based on one or more parameters. For example, the gait coefficient (G) may be based on the calories exerted (E), resting energy requirement (RER), and/or distance traveled (D). The gait coefficient (G) may be adjusted based on the breed of the dog, the size and/or body type of the animal, etc., through the use of an empirically determined multiplier. Using the adjusted gait coefficient, the calories exerted may be determined for each period (e.g., each contiguous period) of gait expression. For periods of rest and/or sleep, G(D) may equal zero, as base RER applies. The calories exerted may be summed for one or more (e.g., all) behaviors across a twenty-four hour period. Such summation may provide a value for the daily calorie expenditure of the animal. The daily calorie expenditure of the animal may be extrapolated for one or more days, weeks, months, etc. The amount of calories exerted during a time period may be determined as the amount of calories required for a present and/or future time period (e.g., same time period). For example, if 400 calories were exerted by the animal during the past twenty-four hours, it may be assumed that 400 calories may be required in the next twenty-four hours. In another example, if the animal's activity results in an additional 400 kcals/day being required by the animal (e.g., required on average over a period of time), it may be determined that 400 kcals/day may be required (e.g., on average) to maintain the weight of the animal over the subsequent period of time until the next calculation is made. Such a determination may be required as activity is variable and the need to account for recent activity and/or the need to revise calculations may be based on recent data.

At 810, the caloric requirement of the animal may be displayed. For example, the caloric requirement may be displayed on a display of a portable electronic device, such as a mobile phone, a tablet, or a mobile phone. The caloric requirement may relate to a daily requirement of the animal, a weekly requirement of the animal, and the like. Subsets of the caloric requirement of the animal may be provided. For example, the daily caloric requirement may be divided into a caloric requirement of the animal for the morning and a caloric requirement for the evening. The user may use such subset data to provide the food required for the animal in the morning and in the evening.

The caloric requirements of the animal may be provided to a veterinarian and/or a pet parent in one or more form factors. For example, notifications of the caloric requirement may be delivered to a portable electronic device, for example, in the form of an electronic mail message sent to a user-specified electronic mail address, a text message sent via SMS (Short Message Service) to a user-specified mobile phone number, a calendar reminder set up by the system in a user-specified calendar, phone calls to a user-specified mobile or landline phone number, messages via a mobile device (e.g., phone, tablet) application (“app”), etc. Upon receiving the caloric requirements, the receiver may provide the required number of calories to the animal. The results of providing the required calories may be received and/or stored for a determination of the success of the animal being fed the required number of calories. Depending on how successful the required number of calories is in reducing health disorders (e.g., obesity), the amount of calories required for the animal may be reduced, increased, or may remain the same.

While the invention has been described with respect to specific examples including presently preferred modes of carrying out the invention, those skilled in the art will appreciate that there are numerous variations and permutations of the above described systems and techniques. It is to be understood that other embodiments may be utilized and structural and functional modifications may be made without departing from the scope of the present invention. Thus, the spirit and scope of the invention should be construed broadly as set forth in the appended claims.

What is claimed is:

1. A method comprising:
receiving, via one or more processors, animal characteristic data relating to an animal;
receiving, via a sensor, movement data of the animal for a first predetermined time period, wherein the movement data comprises at least one of a speed of the animal during the first predetermined time period, a distance traveled by the animal during the first predetermined time period, a location of the animal during the first predetermined time period, or steps taken by the animal during the first predetermined time period;
determining, via one or more processors, an activity level of the animal during the first predefined time period based on the movement data of the animal during the first predetermined time period;
determining, via one or more processors, a caloric requirement of the animal during a second predetermined time period, the caloric requirement of the animal being based on the activity level of the animal during the first predetermined time period and the animal characteristic data of the animal; and
causing the caloric requirement of the animal during the second predetermined time period to be displayed via a display device.

2. The method of claim 1 wherein the sensor is coupled to a collar attached to the animal.

3. The method of claim 1 wherein the first predetermined time period and the second predetermined time period have a same duration.

4. The method of claim 3 wherein the same duration is a duration of twenty-four hours.

5. The method of claim 1 wherein the first predetermined time period and the second predetermined time period have a different duration.

6. The method of claim 1 wherein the animal characteristic data comprises at least one of a weight of an animal, a body type of the animal, a breed of the animal, a body fat index (BFI) of the animal, a body condition score (BCS) of the animal, or a muscle condition score (MCS) of the animal.

7. The method of claim 1 wherein the caloric requirement of the animal is further based on a temperature, heart rate, or respiration rate of the animal.

8. The method of claim 1 wherein the sensor device comprises at least one of an accelerometer, a gyroscope, a magnetometer, or a global positioning system (GPS) device.

9. The method of claim 1 wherein the animal characteristic data further comprises at least one of an activity level, age, or pregnancy condition of the animal.

10. The method of claim 1 wherein the determined caloric requirement of the animal during the second predetermined time period is based on a resting energy requirement exerted by the animal during the first predetermined time period.

11. The method of claim 1 wherein the activity level of the animal during the first predefined time period is based on information including a gait coefficient and a distance travelled by the animal while moving at that gait.

12. The method of claim 1 further comprising determining a trend of caloric requirements of the animal based on the determined caloric requirement of the animal and other caloric requirements determined for the animal.

13. The method of claim 1 wherein at least one of the one or more processors is located at a server.

14. A system for determining calories required of an animal comprising:
a sensor configured to receive movement data of the animal for a first predetermined time period, wherein the movement data comprises at least one of a speed of the animal during the first predetermined time period, a distance traveled by the animal during the first predetermined time period, a location of the animal during the first predetermined time period, or steps taken by the animal during the first predetermined time period; and
one or more processors configured to:
receive animal characteristic data of the animal;
determine an activity level of the animal during the first predefined time period based on the movement data of the animal during the first predetermined time period;
determine a caloric requirement of the animal during a second predetermined time period, the caloric requirement of the animal being based on the activity level of the animal during the first predetermined time period and the animal characteristic data of the animal; and
cause the caloric requirement of the animal during the second predetermined time period to be displayed via a display device.

15. The system of claim 14 wherein the sensor is coupled to a collar attached to the animal.

16. The system of claim 14 wherein the first predetermined time period and the second predetermined time period have a same duration.

17. The system of claim 16 wherein the same duration is a duration of twenty-four hours.

18. The system of claim 14 wherein the first predetermined time period and the second predetermined time period have a different duration.

19. The system of claim 14 wherein the animal characteristic data comprises at least one of a weight of an animal, a body type of the animal, a breed of the animal, a body fat index (BFI) of the animal, a body condition score (BCS) of the animal, or a muscle condition score (MCS) of the animal.

20. The system of claim 14 wherein the caloric requirement of the animal is further based on a temperature, heart rate, or respiration rate of the animal.

21. The system of claim 14 wherein the sensor device comprises at least one of an accelerometer, a gyroscope, a magnetometer, or a global positioning system (GPS) device.

22. The system of claim 14 wherein the animal characteristic data further comprises at least one of an activity level, age, or pregnancy condition of the animal.

23. The system of claim 14 wherein the determined caloric requirement of the animal during the second predetermined time period is based on a resting energy requirement exerted by the animal during the first predetermined time period.

24. The system of claim 14 wherein the activity level of the animal during the first predefined time period is based on a gait of the animal comprising a gait coefficient and a distance traveled by the animal while moving with the gait.

25. The system of claim 14 wherein the one or more processors is configured to determine a trend of caloric requirements of the animal based on the determined caloric requirement of the animal and other caloric requirements determined for the animal.
